(19) United States
(12) Patent Application Publication
(10) Pub. No.: US 2013/0108127 A1
(43) Pub. Date: May 2, 2013

(54) MANAGEMENT OF PATIENT MODEL DATA

(71) Applicants: Thomas Boettger, Heidelberg (DE); Mark Hastenteufel, Heidelberg (DE)

(72) Inventors: Thomas Boettger, Heidelberg (DE); Mark Hastenteufel, Heidelberg (DE)

(21) Appl. No.: 13/286,113

(22) Filed: Oct. 31, 2011

(51) Int. Cl. G06K 9/34; A61B 5/00

(52) U.S. Cl. 382/131; 600/300

(57) ABSTRACT

In order to increase efficiency, an image processing system may create a template identifying volumes of interest to be segmented based on user-specified data relating to a type of treatment and an anatomical site to be treated. The user of the image processing system may create, arrange, view, and manage a patient model including the volumes of interest, reference points, and/or distance measurements based on the arrangement of information using anatomical site rather than using type of imaging modality. The image processing system may segment the identified volumes of interest from image data received from one or more imaging modalities. The image processing system may generate a representation of the patient model including the segmented volumes of interest. The representation of the patient model may be indexed by the volumes of interest.

### FIELD

The present embodiments relate to the creation and management of a patient model.

### BACKGROUND

Patient model creation may be the first step in a treatment planning process, such as treatment planning for radiotherapy. A patient model includes volumes of interest (VOIs) segmented from image data. The volumes of interest may be defined based on a treatment to be performed and an anatomical site, on which the treatment is to be performed (e.g., intensity modulated radiation therapy (IMRT) of a prostate). The image data may include image data generated by one or more different imaging devices (e.g., a computed tomography (CT) device and/or a positron emission tomography (PET) device) at one or more different times.

A user (e.g., a doctor or a nurse) of an image processing system configured to create the patient model must know which VOIs are to be segmented for the treatment case (e.g., the tumor, the liver, the spinal cord, and the skin). The user loads image sets generated from the image data and segments the VOIs. The resultant patient model is indexed by the imaging modality used to generate the image data and the time at which the imaging modality generated the image data.

### SUMMARY

In order to increase the efficiency, an image processing system may create a template identifying volumes of interest to be segmented based on user-specified data relating to a type of treatment and an anatomical site to be treated. The user of the image processing system may create, arrange, view, and manage a patient model based on the arrangement of information using anatomical site rather than using type of imaging modality. The image processing system may segment the identified volumes of interest from image data received from one or more imaging modalities. The image processing system may generate a representation of the patient model including the segmented volumes of interest. The representation of the patient model may be indexed by the volumes of interest.

In a first aspect, a method for extracting a patient model for planning a treatment procedure includes selecting a treatment procedure and an anatomical site. The method includes establishing, by a processor, a patient model template identifying one or more volumes of interest based on the selected treatment procedure and the selected anatomical site. The method also includes segmenting the one or more volumes of interest from a medical data set. The medical data set is obtained using an imaging modality.

In a second aspect, a system for managing patient model data includes a memory configured to store medical imaging data received from a plurality of imaging modalities, each imaging modality of the plurality of imaging modalities representing an examination object at one or more times. The memory is also configured to store user-specified data including a treatment and an anatomical site. The system includes a processor configured to establish a template including structures to be segmented based on the user-specified data, and configured to guide segmentation of the medical imaging data based on the established template. The system also includes a display configured to display a graphical user interface representing the segmented medical imaging data. The segmented medical imaging data is indexed by the structures.

In a third aspect, a non-transitory computer readable medium that stores instructions executable by a processor to manage patient model data is provided. The instructions include receiving medical imaging data produced by an imaging modality. The method also includes receiving user input identifying a medical procedure and an anatomical site, and identifying a plurality of anatomical segments to be segmented. The identifying is based on the medical procedure and the anatomical site input from the user. The method includes segmenting the identified plurality of anatomical segments from the medical imaging data and displaying a representation of the segmented medical imaging data. The representation of the segmented medical imaging data is indexed by the plurality of anatomical segments.

### BRIEF DESCRIPTION OF THE DRAWINGS

FIG. 1 shows one embodiment of an imaging system;

FIG. 2 shows an imaging system including one embodiment of an imaging device;

FIG. 3 shows a data-centric representation of image data;

FIG. 4 shows a flowchart of one embodiment of a method for extracting a patient model for planning a treatment procedure; and

FIG. 5 shows one embodiment of a patient-centric representation of image data.

### DETAILED DESCRIPTION OF THE DRAWINGS

In order to extract a patient model that aids a clinical user in solving a clinical problem (e.g., the planning of a prescribed treatment procedure), the clinical user selects a treatment and an anatomical site. A computer system automatically creates an empty patient model stub with a minimum number of structures (e.g., volumes of interest such as a tumor and/or organs, distance lines, reference points, and/or other kinds of measurements) to be segmented based on the user selected treatment and anatomical site. The clinical user or processor segments at least the minimum number of structures defined by the empty patient model from image data obtained using one or more imaging modalities to fill the empty patient model stub. The computer system generates a graphical user interface that provides a patient-centric representation of the image data indexed by the segmented structures. The clinical user may filter for certain modalities and/or filter for certain time points in order to view desired structure instances.

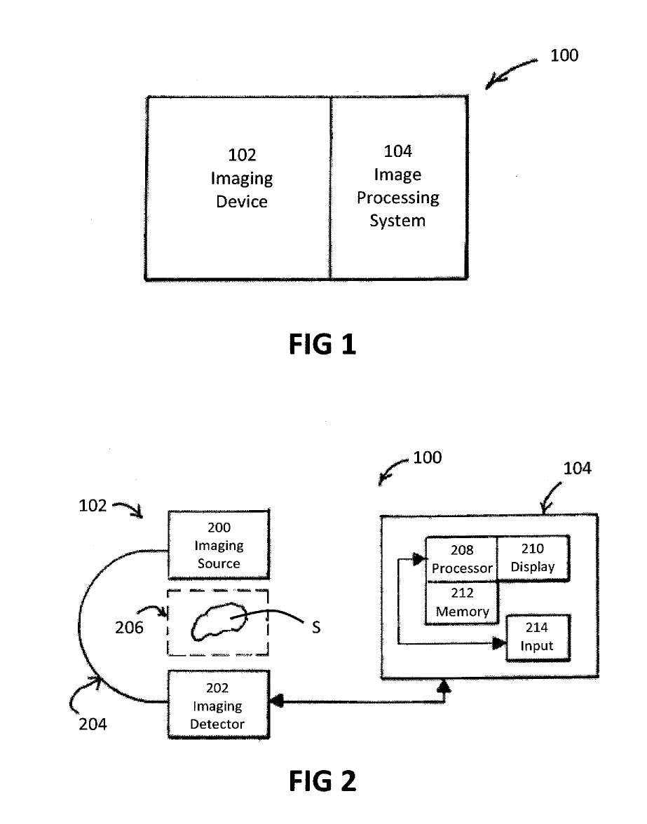

FIG. 1 shows one embodiment of an imaging system 100. The imaging system is representative of an imaging modality. The imaging system 100 may include one or more imaging devices 102 and an image processing system 104. A two-dimensional (2D) or a three-dimensional (3D) (e.g., volumetric) image dataset may be acquired using the imaging system 100. The 2D image data set or the 3D image data set may be obtained contemporaneously with the planning and execution of a medical treatment procedure or at an earlier time. Additional, different, or fewer components may be provided.

The imaging device 102 is one or more of a computed tomography (CT) system, a magnetic resonance imaging (MRI) system, an ultrasound system, a positron emission tomography (PET) system, a single photon emission computed tomography (SPECT) system, an angiography system, a fluoroscopy, an x-ray system, any other now known or later developed imaging systems, or a combination thereof. The image processing system 104 is a workstation, a processor of the imaging device 102, or another image processing device. The imaging system 100 may be used to create a patient model for the planning of the medical treatment procedure (e.g., treatment planning for radiotherapy, interventional oncological ablation procedures, or any navigated, image-guided surgery). For example, the image processing system 104 is a workstation for treatment planning for radiotherapy of a prostate using data from the imaging device 102. The patient model may be created from data generated by the one or more imaging devices 102 (e.g., a CT device, a PET device, and/or an MRI device). The workstation 104 receives data representing the prostate and tissue surrounding the prostate generated by the one or more imaging devices 102.

FIG. 2 shows the imaging system 100 including one embodiment of the imaging device 102. The imaging device 102 is shown in FIG. 2 as a C-arm x-ray device. The imaging device 102 may include an energy source 200 and an imaging detector 202 connected together by a C-arm 204. Additional, different, or fewer components may be provided. In other embodiments, the imaging device 102 may be, for example, a gantry-based CT device, an MRI device, an ultrasound device, a PET device, an angiography device, a fluoroscopy device, another x-ray device, any other now known or later developed imaging devices, or a combination thereof.

The energy source 200 and the imaging detector 202 may be disposed opposite each other. For example, the energy source 200 and the imaging detector 202 may be disposed on diametrically opposite ends of the C-arm 204. In another example, the energy source 200 and the imaging detector 202 are connected inside a gantry. A region 206 to be examined (e.g., of a patient) is located between the energy source 200 and the imaging detector 202. The size of the region 206 to be examined may be defined by an amount, a shape, or an angle of radiation. The region 206 to be examined may include one or more structures S (e.g., one or more volumes of interest, such as the prostrate, a tumor, and surrounding tissue), to which the medical treatment procedure is to be or not be applied (e.g., radiotherapy). The region 206 may be all or a portion of the patient. The region 206 may or may not include a surrounding area. For example, the region 206 to be examined may include the prostate, the tumor, at least a portion of the spinal cord, at least a portion of the bladder, and/or other organs or body parts in the surrounding area of the tumor.

The energy source 200 may be a radiation source such as, for example, an x-ray source. The energy source 200 may emit radiation to the imaging detector 202. The imaging detector 202 may be a radiation detector such as, for example, a digital-based x-ray detector or a film-based x-ray detector. The imaging detector 202 may detect the radiation emitted from the energy source 200. Data is generated based on the amount or strength of radiation detected. For example, the imaging detector 202 detects the strength of the radiation received at the imaging detector 202 and generates data based on the strength of the radiation. The data may be considered imaging data as the data is used to then generate an image. Image data may also include data for a displayed image. In an alternate embodiment, the energy source 200 is a magnetic resonance source or an ultrasound source. In yet other embodiments, the energy source 200 is a radioactive agent provided within the patient.

The data may represent a two-dimensional (2D) or three-dimensional (3D) region, referred to herein as 2D data or 3D data. For example, the C-arm x-ray device 102 may be used to obtain 2D data or CT-like 3D data. A computer tomography (CT) device may obtain 2D data or 3D data. In another example, a fluoroscopy device may obtain 3D representation data. In another example, an ultrasound device may obtain 3D representation data by scanning the region 206 to be examined. The data may be obtained from different directions. For example, the imaging device 102 may obtain data representing sagittal, coronal, or axial planes or distribution.

The imaging device 102 may be communicatively coupled to the image processing system 104. The imaging device 102 may be connected to the image processing system 104, for example, by a communication line, a cable, a wireless device, a communication circuit, and/or another communication device. For example, the imaging device 102 may communicate the data to the image processing system 104. In another example, the image processing system 104 may communicate an instruction such as, for example, a position or angulation instruction to the imaging device 102. All or a portion of the image processing system 104 may be disposed in the imaging device 102, in the same room or different rooms as the imaging device 102, or in the same facility or in different facilities as the imaging device 102.

In one embodiment, a plurality of imaging devices 102 (e.g., the C-arm x-ray device 102 and a PET device) is communicatively coupled to the image processing system 104 by the same or different communication paths. All or some imaging devices 102 of the plurality of imaging devices 102 may be disposed in the same room or same facility. In one embodiment, each imaging device 102 of the plurality of imaging devices 102 may be disposed in a different room. All or a portion of the image processing system 104 may be disposed in one imaging device 102 of the plurality of imaging devices 102. The image processing system 104 may be disposed in the same room or facility as one or more imaging devices 102 of the plurality of imaging devices 102. In one embodiment, the image processing system 104 and the plurality of imaging devices 102 may each be disposed in different rooms or facilities. The image processing system 104 may represent a plurality of image processing systems associated with the plurality of imaging devices 102.

In the embodiment shown in FIG. 2, the image processing system 104 includes a processor 208, a display 210 (e.g., a monitor), and a memory 212. Additional, different, or fewer components may be provided. For example, the image processing system 104 may include an input device 214, a printer, and/or a network communications interface.

The processor 208 is a general processor, a digital signal processor, an application specific integrated circuit, a field programmable gate array, an analog circuit, a digital circuit, another now known or later developed processor, or combinations thereof. The processor 208 may be a single device or a combination of devices such as, for example, associated with a network or distributed processing. Any of various processing strategies such as, for example, multi-processing, multi-tasking, and/or parallel processing may be used. The processor 208 is responsive to instructions stored as part of software, hardware, integrated circuits, firmware, microcode or the like.

The processor 208 may generate an image from the data. The processor 208 processes the data from the imaging device 102 and generates an image based on the data. For example, the processor 208 may generate one or more fluoroscopic images, top-view images, in-plane images, orthogonal images, side-view images, 2D images, 3D representations (i.e., renderings), progression images, multi-planar reconstruction images, projection images, or other images from the data. In another example, a plurality of images may be generated from data detected from a plurality of different positions or angles of the imaging device 102 and/or from a plurality of imaging devices 102.

The processor 208 may generate a 2D image from the data. The 2D image may be a planar slice of the region 206 to be examined. For example, the C-arm x-ray device 102 may be used to detect data that may be used to generate a sagittal image, a coronal image, and an axial image. The sagittal image is a side-view image of the region 206 to be examined. The coronal image is a front-view image of the region 206 to be examined. The axial image is a top-view image of the region 206 to be examined.

The processor may generate a 3D representation from the data. The 3D representation illustrates the region 206 to be examined. The 3D representation may be generated by combining 2D images obtained by the imaging device 102 from given viewing directions. For example, a 3D representation may be generated by analyzing and combining data representing different planes through the patient, such as a stack of sagittal planes, coronal planes, and/or axial planes. Additional, different, or fewer images may be used to generate the 3D representation. Generating the 3D representation is not limited to combining 2D images. For example, any now known or later developed method may be used to generate the 3D representation.

The processor 208 may display the generated images on the monitor 210. For example, the processor 208 may generate the 3D representation and communicate the 3D representation to the monitor 210. The processor 208 and the monitor 210 may be connected by a cable, a circuit, other communication coupling or a combination thereof. The monitor 210 is a monitor, a CRT, an LCD, a plasma screen, a flat panel, a projector or another now known or later developed display device. The monitor 210 is operable to generate images for a two-dimensional view or a rendered three-dimensional representation. For example, a two-dimensional image representing a three-dimensional volume through rendering is displayed.

The processor 208 may communicate with the memory 212. The processor 208 and the memory 212 may be connected by a cable, a circuit, a wireless connection, other communication coupling, or a combination thereof. Images, data, and other information may be communicated from the processor 208 to the memory 212 for storage, and/or the images, the data, and the other information may be communicated from the memory 212 to the processor 208 for processing. For example, the processor 208 may communicate the generated images, image data, or other information to the memory 212 for storage.

The memory 212 is a computer readable storage media. The computer readable storage media may include various types of volatile and non-volatile storage media, including but not limited to random access memory, read-only memory, programmable read-only memory, electrically programmable read-only memory, electrically erasable read-only memory, flash memory, magnetic tape or disk, optical media and the like. The memory 212 may be a single device or a combination of devices. The memory 212 may be adjacent to, part of, networked with and/or remote from the processor 208.

The imaging system 100 may be used to create a patient model for treatment planning for radiotherapy, for example. The patient model may include any kind of measurement data derived from the data generated by the imaging device 102. The measurement data may include volumes of interest (VOIs), reference points, distance measurements, and/or any other functional measurements. For example, the patient model may include segmented images of the one or more structures S at a plurality of time points (e.g., two time points) using the one or more imaging devices 102 (e.g., the C-arm x-ray device and a PET device). A user of the imaging system 100 may segment 2D images or 3D representations (e.g., partitioning the images into multiple segments or sets of pixels) generated by the processor 208 or the image data generated by the imaging device 102. Alternatively or additionally, the processor 208 may automatically segment the 2D images or the 3D representations generated by the processor 208 or the data generated by the imaging device 102. The processor 208 may segment the 2D images, the 3D representations, or the data using segmentation tools and/or algorithms stored in the memory 212 or another memory. For example, the processor 208 may segment the 2D images, the 3D representations, or the data using contouring or delineation tools stored in the memory 212. In one embodiment, the user may create the segmented images of the one or more structures S by manual contour drawing on the 2D images generated by the processor 208. The user may draw the contours delineating the one or more structures S from the 2D images directly on the display 210 or by using the input device 214.

In the prior art, clinical segmentation goals may be data or structure-centric. The user may know which anatomical structures are to be segmented for a treatment case (e.g., a combination of a treatment technique and an anatomical site such as a combination of intensity modulated radiation therapy (IMRT) and a prostate). The user loads image sets from the memory 212. For each of the image sets, the user creates segmented images of at least some of the one or more structures S in at least some images of the image set. The segmented image information is stored as a structure set (e.g., a set of the one or more structures S segmented from the image set) in the memory 212.

For example, the patient model for the IMRT of the prostate may be formed from a plurality of images (e.g., at two time points) generated using the C-arm x-ray device 102 (e.g., a plurality of CT images; a CT image set) shown in FIG. 2 and an image generated using a PET device (e.g., a PET image). Based on the treatment case (e.g., IMRT of the prostate), the user may know that the tumor, the liver, the spinal cord and the skin are to be segmented in order to form the patient model for the IMRT of the prostate and plan the IMRT of the prostate.

The user loads a first CT image of the CT image set (e.g., a CT image at a first time point), creates a first structure set including segmented images of the tumor, the liver, the spinal cord, and the skin, and stores the first structure set in the memory 212. The user loads a second CT image of the CT image set (e.g., a CT image at a second time point), creates a second structure set including segmented images of the tumor, the liver, and the skin, and stores the second structure set in the memory 212. The user loads the PET image (e.g., a PET image at the first time point), creates a third structure set including a segmented image of the tumor, and stores the third structure set in the memory 212.

A textual representation of the patient model including the first structure set, the second structure set, and the third structure set may be displayed in a data-centric view on the display 210 or another display:

StructureSet1:CT:time1

### ->Tumor

### ->Liver

->Spinal cord

### ->Skin

StructureSet2:CT:time2

### ->Tumor

### ->Liver

### ->Skin

StructureSet3:PET:time1

### ->Tumor

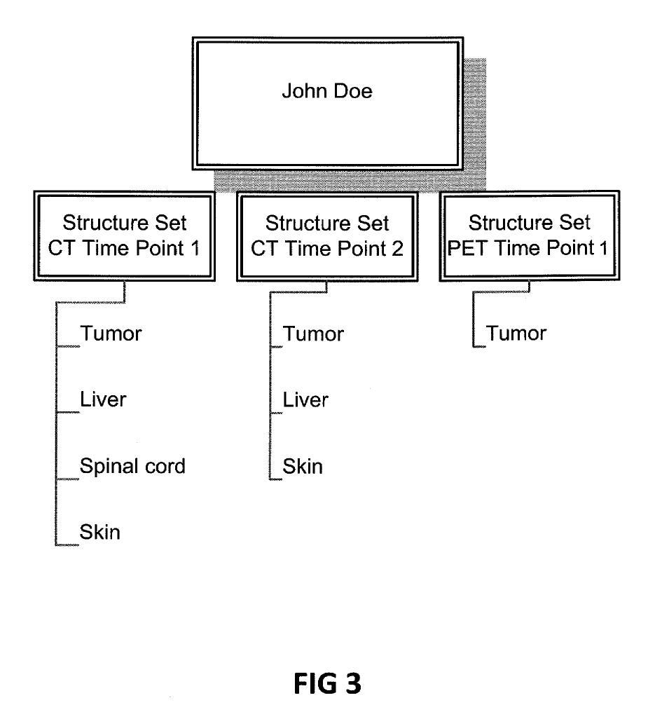

FIG. 3 represents this data-centric arrangement for display. The user may select one of the segmented images (e.g., the segmented image of the tumor in the first structure set) using the input device, for example, and the display 210 displays the selected segmented image to aid in the planning of the IMRT of the prostate.

A representation of the patient model may be displayed on the display 210 as a graphical user interface (GUI). FIG. 3 shows an example of a GUI displayed on the display 210 that represents the patient model including the first structure set, the second structure set, and the third structure set. The user may select one of the segmented images (e.g., the segmented image of the tumor in the first structure set) on the GUI to display the segmented image on the display 210. The user may select the segmented image using the input device 214. In one embodiment, the display 210 may be a touch screen, and the user may select the segmented image directly on the display 210.

With the data-centric approach, the user must know which structures are to be created (e.g., segmented) for a certain treatment case and in which structure set the relevant structure instances are located. This leads to solution oriented planning (e.g., segment the tumor, the liver, the spinal cord, and skin in order to plan IMRT of the prostate). The data-centric approach may make arranging, viewing, and managing the patient model time consuming.

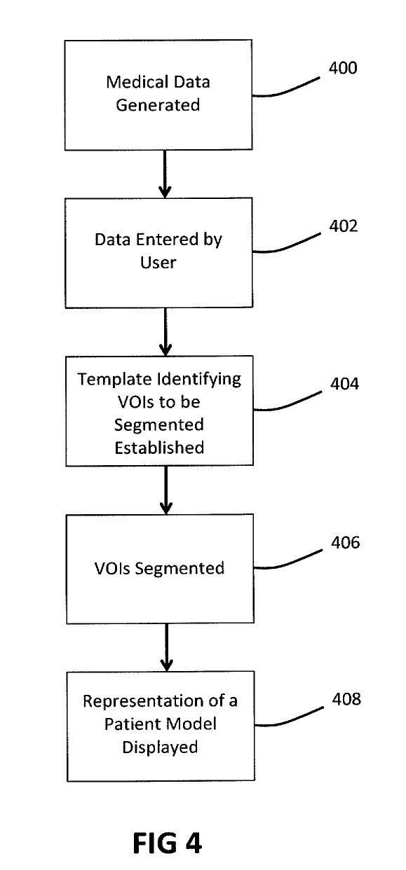

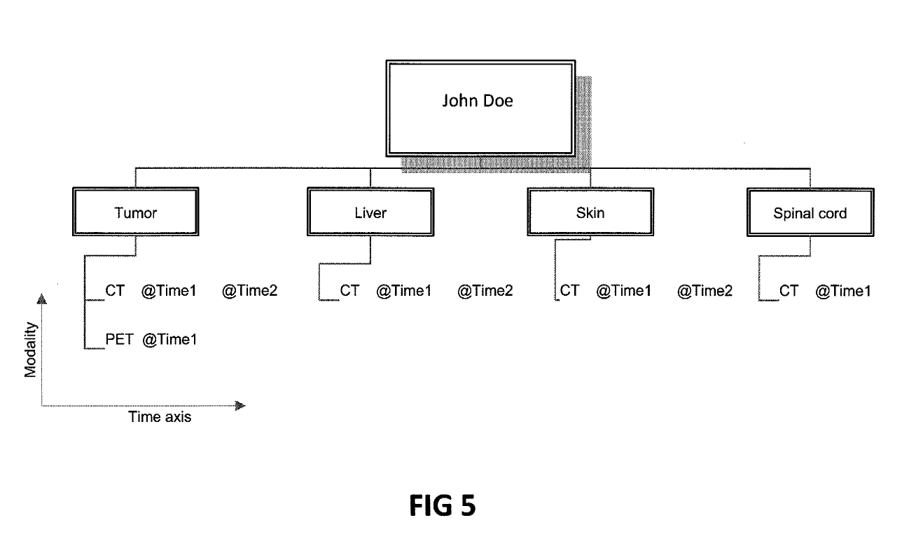

In the present embodiments, the clinical segmentation goals are problem oriented or patient-centric. FIG. 4 shows a flowchart of one embodiment of a method for extracting a patient model for planning a treatment procedure. The method may be performed using the imaging system 100 shown in FIGS. 1 and 2 or another imaging system. The method is implemented in the order shown, but other orders may be used. Additional, different, or fewer acts may be provided. Similar methods may be used for extracting the patient model for planning the treatment procedure.

In act 400, one or more imaging modalities may generate medical data. The one or more imaging modalities may transmit the medical data to an image processing system. The one or more imaging modalities may include any number of medical imaging devices including, for example, a C-arm x-ray device, a gantry-based CT device, an MRI device, an ultrasound device, a PET device, a SPECT device, an angiography device, a fluoroscopy device, another x-ray device, any other now known or later developed imaging devices, or a combination thereof. The medical imaging data may be 2D data or 3D data. For example, a CT device may obtain 2D data or 3D data. In another example, a fluoroscopy device may obtain 3D representation data. In another example, an ultrasound device may obtain 3D representation data by scanning a region to be examined. The medical data may be obtained from different directions. For example, the one or more imaging modalities may obtain sagittal, coronal, or axial data.

In act 402, a user of the image processing system enters data (e.g., user-specified data) into the image processing system using, for example, an input device (e.g., a keyboard or a mouse) of the image processing system. The user-specified data may identify a medical procedure or treatment to be performed (e.g., a treatment technique, such as IMRT) and an anatomical site of a patient to be treated (e.g., the prostate). The user may use the keyboard to enter the data into a graphical user interface displayed on the display. Alternatively, the user may use the mouse to select the treatment technique and/or the anatomical site from drop-down boxes or options displayed on the graphical user interface. Other forms of data entry may be used.

In act 404, a template (e.g., an empty patient model) identifying volumes of interest (VOIs) (e.g., structures) to be segmented for the patient model may be generated or established based on the user-specified data. Data identifying VOIs to be segmented (e.g., the tumor, the liver, the spinal cord, and the skin) for a plurality of combinations of treatment techniques and anatomical sites (e.g., IMRT of the prostate) may be stored in a memory of the image processing system. In one embodiment, the data corresponding to the VOIs to be segmented for the plurality of different treatment techniques may be stored in a look-up table in the memory. For example, the user may enter “IMRT” and “prostate” in act 402, and a processor of the image processing system may compare a combination of “IMRT” and “prostate” to combinations in the look-up table. The look-up table may return “Tumor,” “Liver,” “Spinal cord,” and “Skin,” as VOIs to be segmented. The processor generates or establishes the empty patient model based on the returned VOIs to be segmented. In one embodiment, the empty patient model may be established from scratch. The generated or established template acts as an outline identifying the VOIs to be segmented by the processor and/or the user. The generated or established template may guide segmentation by providing the VOIs to be segmented to the processor or by indicating the VOIs to be segmented to the user.

In act 406, the VOIs are segmented from data generated by at least one of the one or more imaging modalities (e.g., the CT device). The data generated by the CT device may be processed and displayed at the image processing system as a 2D CT image or a 3D CT representation including the tumor, the liver, the spinal cord, and the skin of the patient.

The user may segment the imaging data generated by the at least one imaging modality by drawing contours on the 2D CT image to segment the tumor, the liver, the spinal cord, and the skin from the 2D CT image. Other segmentation methods using, for example, contouring or delineation tools and/or algorithms may be used to segment the imaging data. Other VOIs not identified in act 404 may also be segmented from the 2D CT imaging data. The VOIs may also be segmented from 2D CT data generated at a different time point and/or from data generated by other imaging modalities (e.g., the PET device) of the one or more imaging modalities.

In one embodiment, the patient model includes a plurality of VOIs (e.g., the tumor, the liver, the spinal cord, and the skin) and a plurality of VOI instances (e.g., segmented from imaging data received from a plurality of imaging modalities at a plurality of time points). For example, the patient model may include: segmented CT images of the tumor at a first time point and a second time point, and a segmented PET image of the tumor at the first time point; segmented CT images of the liver at the first time point and the second time point; a segmented CT image of the spinal cord at the first time point; and segmented CT images of the skin at the first time point and the second time point. Other sub-divisions of the data for each VOI may be provided. In one embodiment, the data structure for each VOI is of the same format. In other embodiments, different VOIs may have different formats.

The user may be assisted by the format of the presentation. By having an anatomy-based data organization, the user may walk through the segmentations to be performed, either for segmenting or confirming proper processor based segmentation. The user may sequentially deal with all or the desired data for a given anatomy or VOI. This may allow for comparison of the segmentations for diagnosis or segmentation performance.

In act 408, a representation of the patient model (e.g., a representation of the segmented imaging data) may be displayed. The representation of the patient model may include the plurality of VOIs and the plurality of VOI instances. The patient model may be indexed by the plurality of VOIs. In one embodiment, the representation of the patient model is a textual representation that may be displayed in a patient-centric view on the display:

### Filter: ALL|CT|MRI|PET

### Filter: All|Time1|Time2|Time3

### ->Tumor

->Tumor_CT_time1

->Tumor_PET_time1

->Tumor_CT_time2

### ->Liver

->Liver_CT_time1

->Liver_CT_time2

->Spinal cord

->Spinal_cord_CT_time1

### ->Skin

->Skin_CT_time1

->Skin_CT_time2

The user may select one of the segmented images (e.g., the segmented CT image of the tumor at the first time point, labeled “Tumor_CT_time1”) using the input device, for example, and the display displays the selected segmented image to aid in the planning of the IMRT of the prostate. In other embodiments, the user may filter for certain imaging modalities and/or time points in order to display a plurality of the segmented images together. For example, the user may select “Filter: CT” to instruct the processor to display the segmented CT image of the tumor at the first time point, the segmented CT image of the tumor at the second time point, the segmented CT image of the liver at the first time point, the segmented CT image of the liver at the second time point, the segmented CT image of the spinal cord at the first time point, the segmented CT image of the skin at the first time point, and the segmented CT image of the skin at the second time point together.

FIG. 5 shows another example of the representation of the patient model indexed by the VOIs. The patient model is represented by a GUI. The user may select one of the segmented images (e.g., the segmented CT image of the tumor at the first time point) on the GUI to display the one segmented image on the display. The user may select the one segmented image using the input device. In one embodiment, the display may be a touch screen, and the user may select the one segmented image directly on the display.

In other embodiments, FIG. 5 with or without the instance labels is displayed as the template to be filled. For example, the VOIs of interest are displayed for a given patient without any instance information. As another example, the VOIs of interest and instances of interest (e.g., CT and PET images desired for prostate treatment planning) are displayed prior to associating data or segmented images with the specific instances. In alternative embodiments, the model is not displayed before linking the instances, but instead used to indicate sequentially to the user each of the instances needed to complete the model.

The present embodiments provide efficient, problem-oriented guidance for identifying the VOIs to be segmented for planning different treatment techniques for different anatomical sites. The user may create segmentations of the same VOI using different imaging modalities at different time points, and a patient-centric view of a patient model may always be displayed. The patient does not have to know which VOIs are to be segmented for the different treatment techniques for the different anatomical sites. The patient-centric view may allow the user to more easily arrange, view, and manage the segmented VOIs for treatment planning.

While the present invention has been described above by reference to various embodiments, it should be understood that many changes and modifications can be made to the described embodiments. It is therefore intended that the foregoing description be regarded as illustrative rather than limiting, and that it be understood that all equivalents and/or combinations of embodiments are intended to be included in this description.

1. A method for extracting a patient model for planning a treatment procedure, the method comprising:
selecting the treatment procedure and an anatomical site;
establishing, by a processor, a patient model template identifying one or more volumes of interest based on the selected treatment procedure and the selected anatomical site; and
segmenting the one or more volumes of interest from a medical data set, the medical data set obtained using an imaging modality.

2. The method of claim 1, further comprising segmenting another volume of interest from the medical data set.

3. The method of claim 1, further comprising displaying a representation of the patient model, the representation of the patient model being indexed by the one or more volumes of interest.

4. The method of claim 1, wherein the medical data set is a first data set obtained at a first time using the imaging modality,
wherein segmenting the one or more volumes of interest comprises segmenting the first data set obtained at the first time, and
wherein the method further comprises segmenting at least some of the one or more volumes of interest from a second data set, the second data set being obtained at a second time using the imaging modality.

5. The method of claim 4, wherein the imaging modality is a first imaging modality, and
wherein the method further comprises segmenting at least some of the one or more volumes of interest from a third data set, the third data set being obtained at the first time using a second imaging modality.

6. The method of claim 5, further comprising filtering the first data set, the second data set, and the third data set as a function of an imaging modality used, a volume of interest of the one or more volumes of interest, or a time.

7. The method of claim 6, wherein the first data set, the second data set, and the third data set are filtered as a function of the volume of interest, and
wherein the method further comprises displaying an image of the volume of interest based on the filtered first data set, the filtered second data set, and the filtered third data set.

8. The method of claim 1, wherein the one or more segmented volumes of interest are represented as one or more reference points, one or more distance lines, or one or more reference points and one or more distance lines.

9. A system for managing patient model data, the system comprising:
a memory configured to store:
medical imaging data received from a plurality of imaging modalities, each imaging modality of the plurality of imaging modalities representing an examination object at one or more times; and
user-specified data including a treatment and an anatomical site;
a processor configured to:
establish a template comprising structures to be segmented based on the user-specified data; and
guide segmentation of the medical imaging data based on the established template; and
a display configured to display a graphical user interface representing the segmented medical imaging data, the segmented medical imaging data being indexed by the structures.

10. The system of claim 9, wherein the display is further configured to simultaneously display a plurality of images of one of the structures based on the segmented medical imaging data.

11. The system of claim 9, wherein the processor is further configured to filter the segmented medical imaging data based on a filtering criteria, the graphical user interface enabling a user to specify the filtering criteria.

12. The system of claim 11, wherein the display is further configured to simultaneously display one or more images of one of the structures based on the filtered segmented medical imaging data.

13. The system of claim 11, wherein the filtering criteria is based on an imaging modality used, a segmented structure, or a time of imaging.

14. The system as claimed in claim 9, wherein the plurality of imaging modalities comprises at least two of a computed tomography (CT) device, a magnetic resonance tomography (MRT) device, a positron emission tomography (PET) device, and an ultrasound device.

15. In a non-transitory computer readable medium that stores instructions executable by a processor to manage patient model data, the instructions comprising:
receiving medical imaging data produced by an imaging modality;
receiving user input identifying a medical procedure and an anatomical site;
identifying a plurality of anatomical segments to be segmented, the identifying being based on the medical procedure and the anatomical site input from the user;
segmenting the identified plurality of anatomical segments from the medical imaging data; and
displaying a representation of the segmented medical imaging data, the representation of the segmented medical imaging data being indexed by the plurality of anatomical segments.

16. The non-transitory computer readable medium of claim 15, wherein the representation of the segmented medical imaging data is further indexed by an imaging modality used.

17. The non-transitory computer readable medium of claim 15, wherein the representation of the segmented medical imaging data is further indexed by a time of imaging.

18. The non-transitory computer readable medium of claim 15, further comprising:
receiving a user-defined filter criteria; and
filtering the segmented medical imaging data based on the user-defined filter criteria.

19. The non-transitory computer readable medium of claim 19, further comprising displaying one or more images based on the filtered medical imaging data.

20. The non-transitory computer readable medium of claim 15, wherein identifying the plurality of anatomical segments to be segmented comprises:
comparing data including a combination of the medical procedure and the anatomical site input from the user to a look-up table stored in a memory; and
outputting data representing the anatomical segments to be segmented based on the comparison.
